(19) United States
(12) Patent Application Publication
(10) Pub. No.: US 2014/0113360 A1
(43) Pub. Date: Apr. 24, 2014

(54) CELL CHIP CONTAINER FOR STORING CELL CHIP

(71) Applicant: SAMSUNG ELECTRO-MECHANICS CO., LTD., Suwon (KR)

(72) Inventors: Dong Woo LEE, Suwon (KR); Bo Sung KU, Suwon (KR)

(73) Assignee: SAMSUNG ELECTRO-MECHANICS CO., LTD., Suwon (KR)

(21) Appl. No.: 13/761,793

(22) Filed: Feb. 7, 2013

(30) Foreign Application Priority Data
Oct. 18, 2012 (KR) 10-2012-0116169

(51) Int. Cl. C12M 1/00

(52) U.S. Cl. CPC C12M 21/00
USPC 435/289.1; 435/303.1; 422/547

(57) ABSTRACT

There is provided a cell chip including: a first substrate provided with a culture region receiving a bio-material and a humidity control space storing water for maintenance of humidity; a second substrate coupled to the first substrate; and a first wall member surrounding the culture region.

### CROSS-REFERENCE TO RELATED APPLICATIONS

This application claims the priority of Korean Patent Application No. 10-2012-0116169 filed on Oct. 18, 2012, in the Korean Intellectual Property Office, the disclosure of which is incorporated herein by reference.

### BACKGROUND OF THE INVENTION

1. Field of the Invention

The present invention relates to a cell chip and a container for storing a cell chip, and more particularly, to a cell chip capable of suppressing a phenomenon in which a bio material of the cell chip is dried, and a container for storing a cell chip.

2. Description of the Related Art

The demand for a biomedical apparatus and biotechnology for rapidly diagnosing various human diseases has been recently increased. Therefore, usage of a cell chip capable of simultaneously conducting experiments with a plurality of bio materials and drugs has been gradually increased. Since the cell chip is provided with various bio materials and drugs, results of reactions of a plurality of bio materials and drugs may be observed in a single one experiment.

However, the cell chip having a structure in which only a trace of the bio material is received, may be easily influenced by an external environment, and accordingly, the bio materials and the drugs may be easily dried. In this case, since reliability in experimental results using the cell chip is deteriorated, the development of a technology for constantly maintaining humidity in or around the cell chip is required.

Meanwhile, as related technologies, there are provided patent documents 1 and 2 in the Related Art Document below. Patent document 2 does not disclose any configuration in which humidity is constantly maintained; however, patent document 1 discloses formation of an evaporation controlling section 3 in order to constantly maintain humidity of a culture section 1. However, since the evaporation controlling section 3 is limited to an edge of the culture section 1 in the patent document 1, the humidity in a central portion of the culture section 2 is not constantly maintained.

### RELATED ART DOCUMENT

(Patent Document 1) JP 2000-236869 A
(Patent Document 2) JP 2007-024562 A

### SUMMARY OF THE INVENTION

An aspect of the present invention provides a cell chip capable of having constant humidity maintained in the entire region thereof, and a container for storing the cell chip.

According to an aspect of the present invention, there is provided a cell chip including: a first substrate provided with a culture region receiving a bio-material and a humidity control space storing water for maintenance of humidity; a second substrate coupled to the first substrate; and a first wall member surrounding the culture region.

The first wall member may be formed on the first substrate.

The first wall member may be formed on the second substrate.

The humidity control space may be formed along an edge of the culture region in a lengthwise manner.

The humidity control space may be disposed in a position higher than that of the culture region.

The first wall member may be provided with at least one hole allowing the humidity control space and the culture region to be in communication with each other.

The first wall member may partially surround the culture region.

The culture region may be configured of at least one groove receiving the bio-material.

The cell chip may further include a second wall member surrounding the humidity control space.

The second wall member may be formed on the first substrate.

The second wall member may be formed on the second substrate.

The second wall member may be provided with at least one hole allowing the exterior environment and the humidity control space to be in communication with each other.

The second wall member may partially surround the culture region.

The first wall member may partially surround the culture region, and the second wall member may surround a remaining portion of the culture region.

According to another aspect of the present invention, there is provided a container for storing a cell chip including: a bottom member provided with a mounting space receiving at least one cell chip and a humidity control space storing water for maintenance of humidity; a cover member coupled with the bottom member; and a first wall member surrounding the mounting space.

The first wall member may be formed on the bottom member.

The first wall member may be formed on the cover member.

The humidity control space may be formed along an edge of the mounting space in a lengthwise manner.

The humidity control space may be disposed in a position higher than that of the mounting space.

The first wall member may be provided with at least one hole allowing the humidity control space and the mounting space to be in communication with each other.

The first wall member may partially surround the mounting space.

The container for storing a cell chip may further include a second wall member surrounding the humidity control space.

The second wall member may be formed on the bottom member.

The second wall member may be formed on the cover member.

The second wall member may be provided with at least one hole allowing the exterior environment and the humidity control space to be in communication with each other.

The second wall member may partially surround the culture region.

The first wall member may partially surround the culture region, and the second wall member may surround a remaining portion of the culture region.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is an exploded perspective view of a cell chip according to an embodiment of the present invention;

FIG. 2 is an assembled view of the cell chip shown in FIG. 1;

FIG. 3 is a cross-sectional view taken along line A-A of the cell chip shown in FIG. 2;

FIG. 4 is a cross-sectional view taken along line B-B of the cell chip for explaining air flow in the cell chip shown in FIG. 3;

FIG. 5 is a cross-sectional view taken along line A-A of another embodiment of a first substrate shown in FIG. 1;

FIG. 6 is a cross-sectional view taken along line A-A of another embodiment of a second substrate in the cell chip of FIG. 5;

FIG. 7 is a view showing another embodiment of a first wall member shown in FIG. 1;

FIG. 8 is a view showing another embodiment of the first wall member shown in FIG. 1;

FIG. 9 is an exploded perspective view of a cell chip according to another embodiment of the present invention;

FIG. 10 is an assembled cross-sectional view of the cell chip shown in FIG. 9;

FIG. 11 is a cross-sectional view taken along line C-C of the cell chip shown in FIG. 10;

FIG. 12 is a view showing another embodiment of the first substrate shown in FIG. 9;

FIG. 13 is an exploded perspective view of a container for storing the cell chip according to an embodiment of the present invention;

FIG. 14 is a plan view of the container for storing the cell chip shown in FIG. 13;

FIG. 15 is a cross-sectional view taken along line D-D of the container for storing a cell chip shown in FIG. 13; and

FIG. 16 is a cross-sectional view taken along line D-D of a container for storing a cell chip according to another embodiment of the present invention.

### DETAILED DESCRIPTION OF THE EMBODIMENTS

Hereinafter, embodiments of the present invention will be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art.

In the drawings, the shapes and dimensions of elements may be exaggerated for clarity, and the same reference numerals will be used throughout to designate the same or like elements.

For reference, a substrate described in an embodiment of the present invention refers to a member used for a bio material experiment, and a material thereof is not particularly limited. Therefore, the substrate described below may be formed of silicon, a glass, a metal, or a polymer. Here, the polymer may be polymethylmethacrylate (PMMA), polycarbonate (PC), polystyrene (PS), polypropylene, a cyclic olefin copolymer, polynorbonene, a styrene-butadiene copolymer (SBC), or acrylonitrile butadiene styrene; however, it is not particularly limited.

In addition, a method of manufacturing the substrate is not particularly limited. For example, the substrate may be manufactured with a photo resist process, an etching process, an injection molding process, and the like.

Further, the bio material mentioned in an embodiment of the present invention may refer to various materials. For example, the bio material according to the embodiment of the present invention may be nucleic sequence such as ribonucleic acid (RNA), deoxyribonucleic acid (DNA), or the like, a peptide, a protein, a lipid, an organic or an inorganic chemical molecule, a virus particle, a prokaryotic cell, an organelle, or the like. In addition, the bio material is not limited to a human cell, but may include cells of various kinds of animals and plants.

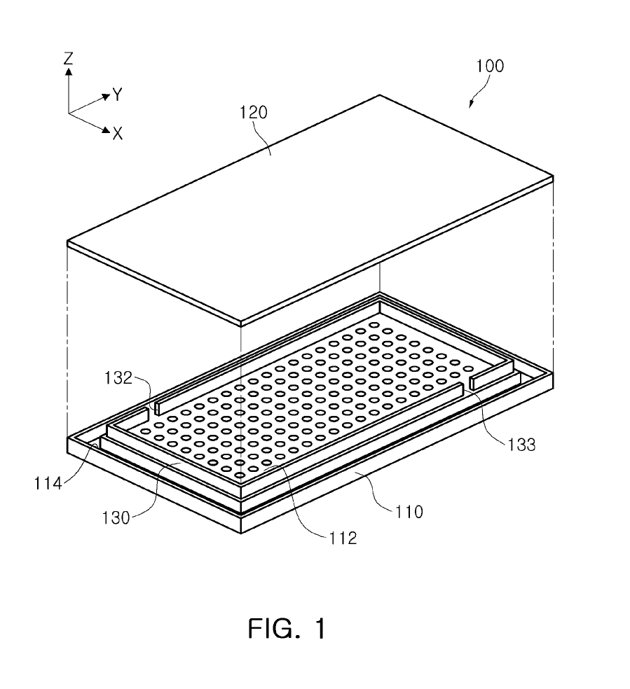

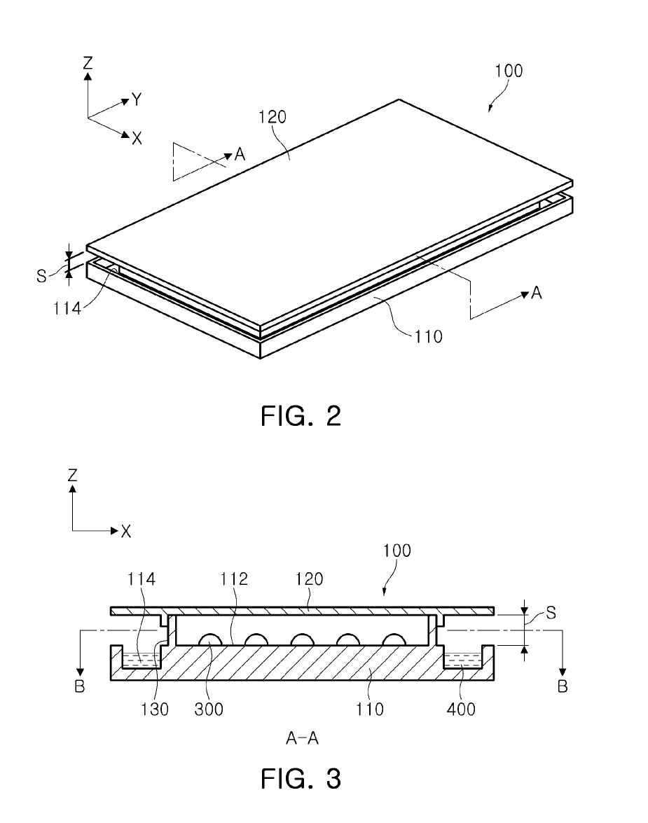

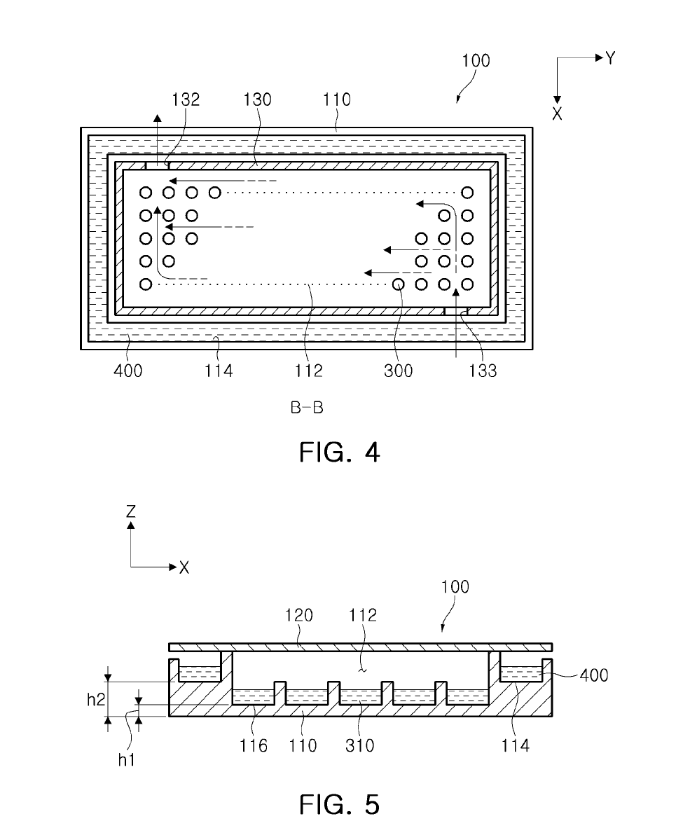

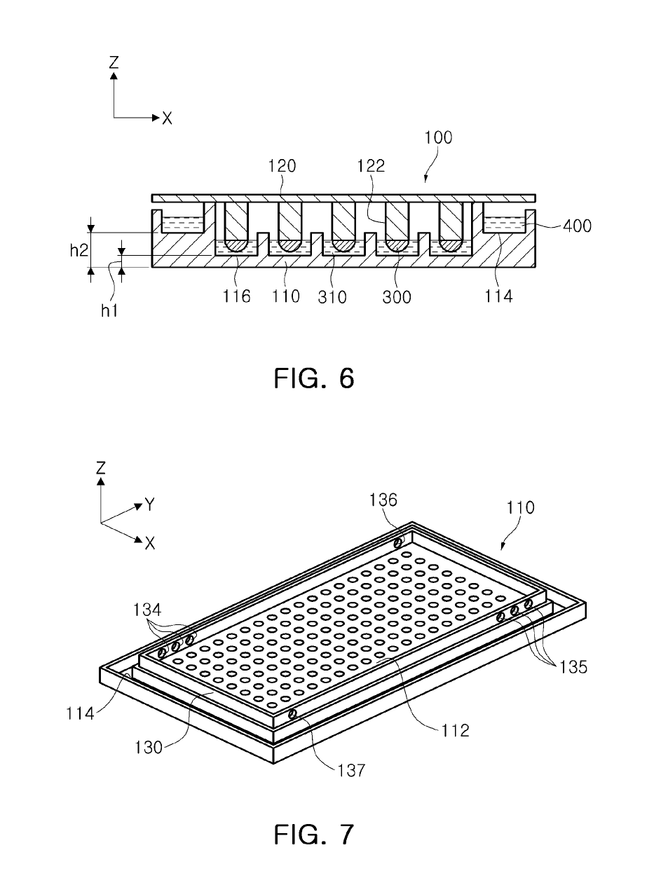

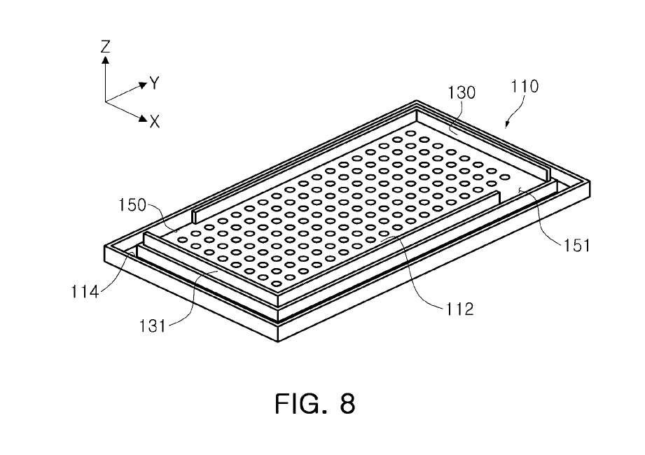

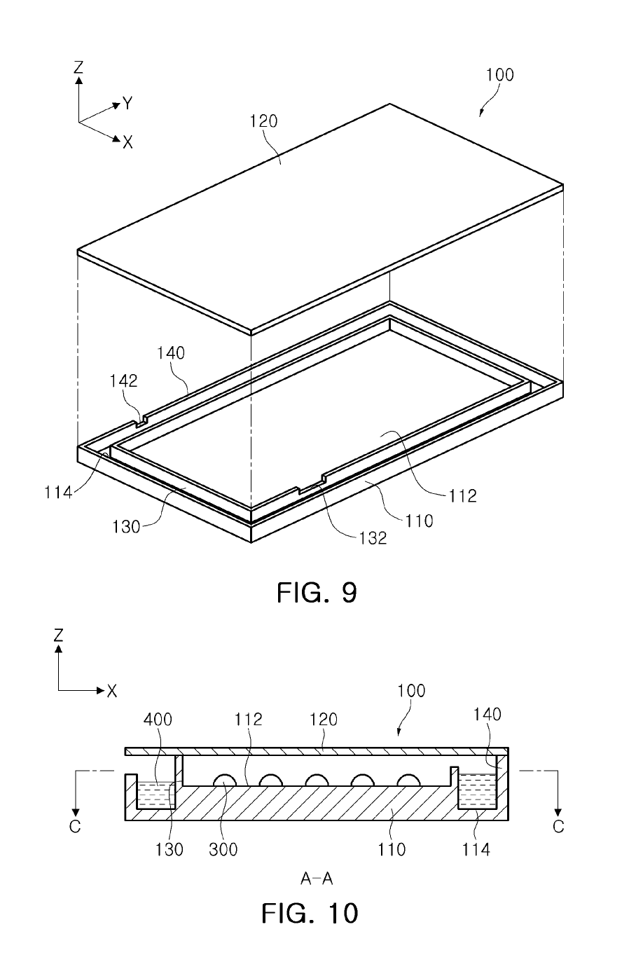

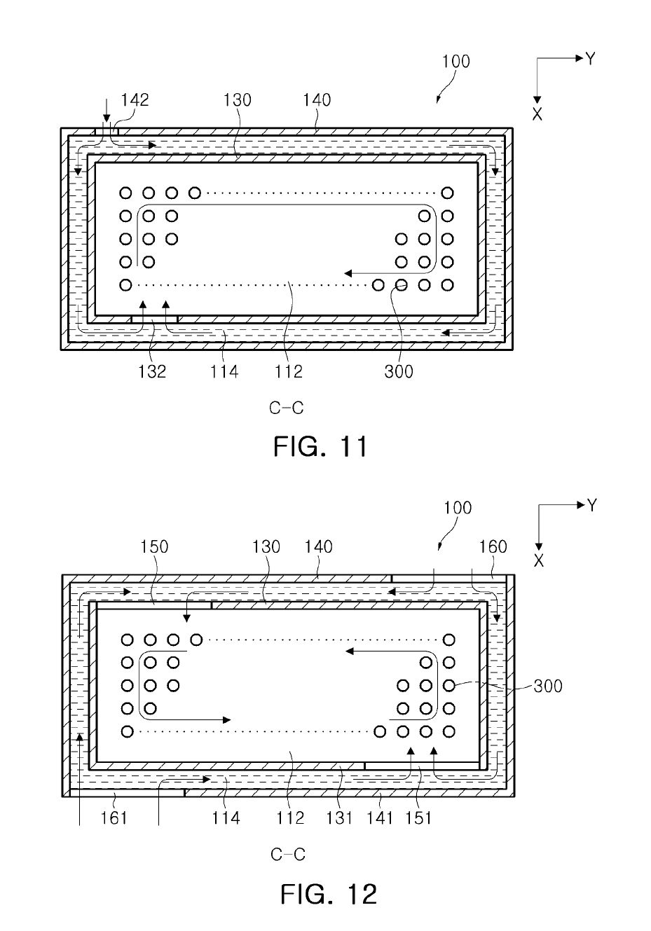

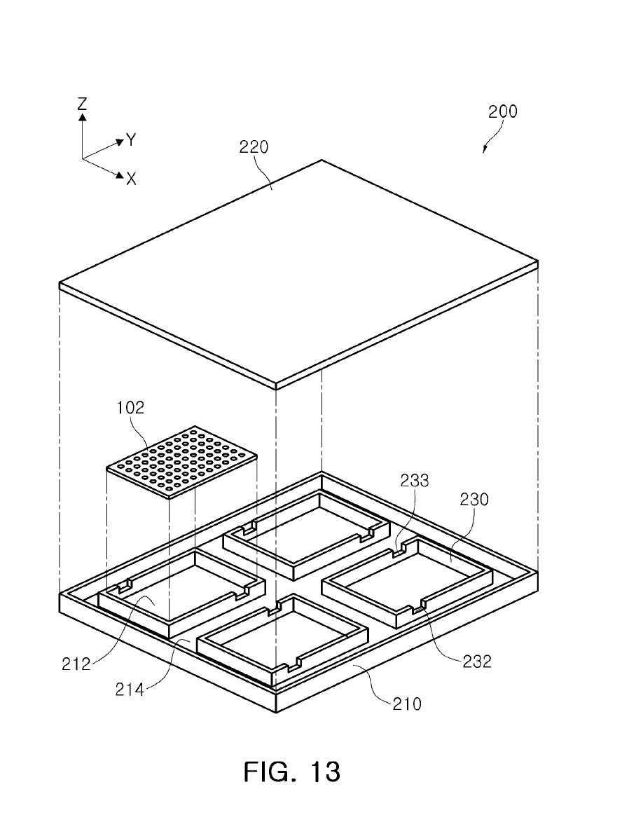

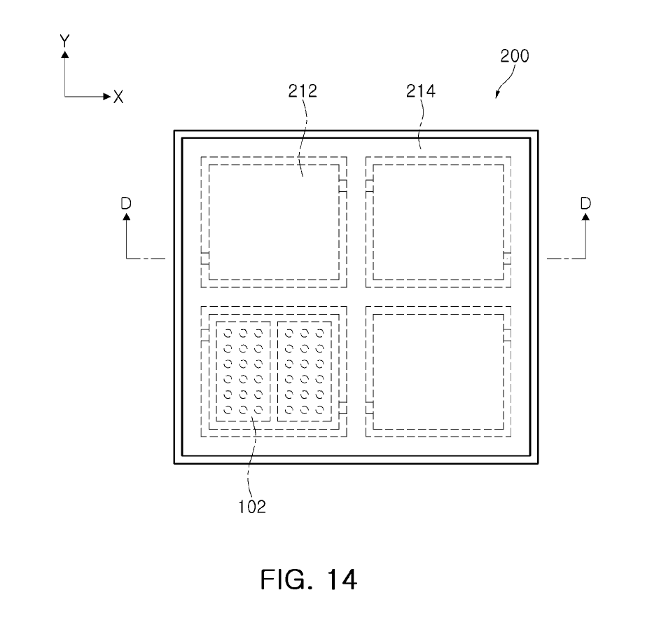

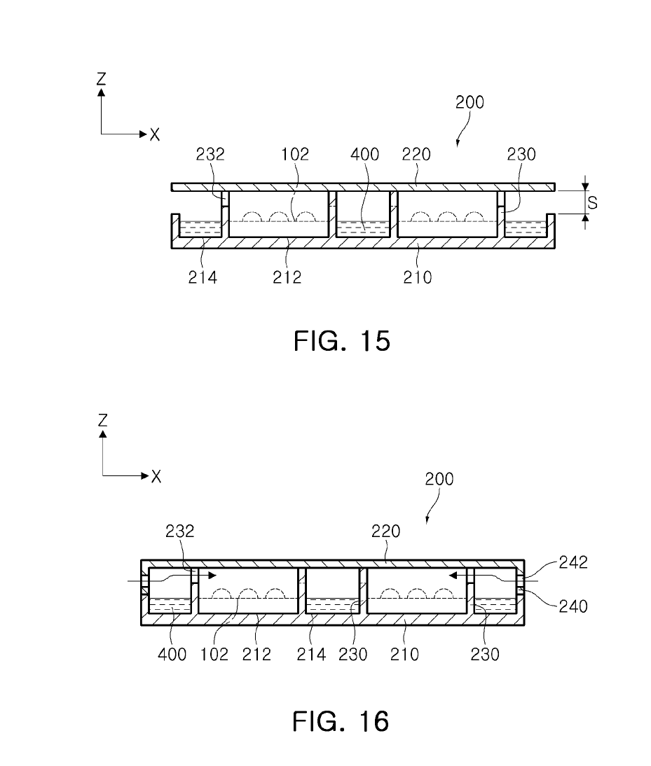

FIG. 1 is an exploded perspective view of a cell chip according to an embodiment of the present invention; FIG. 2 is an assembled view of the cell chip shown in FIG. 1; FIG. 3 is a cross-sectional view taken along line A-A of the cell chip shown in FIG. 2; FIG. 4 is a cross-sectional view taken along line B-B of the cell chip for explaining air flow in the cell chip shown in FIG. 3; FIG. 5 is a cross-sectional view taken along line A-A of another embodiment of a first substrate shown in FIG. 1; FIG. 6 is a cross-sectional view taken along line A-A of another embodiment of a second substrate in the cell chip of FIG. 5; FIG. 7 is a view showing another embodiment of a first wall member shown in FIG. 1; FIG. 8 is a view showing another embodiment of the first wall member shown in FIG. 1; FIG. 9 is an exploded perspective view of a cell chip according to another embodiment of the present invention; FIG. 10 is an assembled cross-sectional view of the cell chip shown in FIG. 9; FIG. 11 is a cross-sectional view taken along line C-C of the cell chip shown in FIG. 10; FIG. 12 is a view showing another embodiment of the first substrate shown in FIG. 9; FIG. 13 is an exploded perspective view of a container for storing the cell chip according to an embodiment of the present invention; FIG. 14 is a plan view of the container for storing the cell chip shown in FIG. 13; FIG. 15 is a cross-sectional view taken along line D-D of the container for storing a cell chip shown in FIG. 13; and FIG. 16 is a cross-sectional view taken along line D-D of a container for storing a cell chip according to another embodiment of the present invention.

The cell chip according to the embodiment of the present invention will be described with reference to FIGS. 1 to 8.

The cell chip 100 according to the embodiment of the present invention may include a first substrate 110 and a second substrate 120. The first substrate 110 may be combined with the second substrate 120 in a vertical direction (a Z axis direction based on FIG. 1), and it may also be separated therefrom as needed.

The first substrate 110 may receive the bio materials or the drugs. To this end, the first substrate 110 may have a culture region 112 to which the bio materials or the drugs may be attached or fixed. More specifically, the culture region 112 may be formed on one surface (an upper surface based on FIG. 1) of the first substrate 110. Here, the culture region 112 may be a plane which does not have a specific shape. However, the culture region 112 may be coated with a specific material as needed. For example, the culture region 112 may be coated with a hydrophilic material so that the bio materials may be easily adhered or fixed thereto. More specifically, a partial region of the culture region 112 to which bio materials are attached may be coated with a hydrophilic material and a remaining region thereof may be coated with a hydrophobic material.

The first substrate 110 may further include a humidity control space 114. More specifically, the humidity control space 114 may be formed on one surface of the first substrate 110, similar to the culture region 112. The humidity control space 114 may be formed at an edge of the culture region 112. More specifically, the humidity control space 114 may be formed along the edge of the culture region 112 in a lengthwise manner. The humidity control space 114 formed as described above may be filled with water for controlling humidity. Therefore, surroundings of the culture region 112 may be maintained at a constant humidity by water in the humidity control space 114.

The first substrate 110 may further include a first wall member 130. The first wall member 130 may surround the culture region 112. Therefore, the first substrate 110 may be divided into the culture region 112 and the humidity control space 114 by the first wall member 130. The first wall member 130 may be provided with at least one or more first cut parts 132 and 133. Here, in the case in which the first wall member 130 is provided with two first cut parts 132 and 133, the first cut parts 132 and the first cut part 133 may be formed so that a distance at which they pass through the culture region 112 is significantly large. Therefore, it may allow air introduced through the first cut parts 132 and 133 to remain in the culture region 112 for an extended period of time.

The second substrate 120 may be coupled to the first substrate 110. More specifically, the second substrate 120 may be disposed above the first substrate 110 and cover one surface of the first substrate 110. To this end, the second substrate 120 may generally have the same size as the first substrate 110.

The first substrate 110 and the second substrate 120 as configured above may be coupled to each other in the vertical direction (a Z axis direction based on FIG. 2) as shown in FIG. 2.

In a state in which the first substrate 110 and the second substrate 120 are coupled to each other, an edge of the first substrate 110 and an edge of the second substrate 120 may have a predetermined open space S.

The open space S may be formed on the humidity control space 114. The open space S formed as described above may be used as a buffer space preventing dry external air from being directly introduced to the culture region 112. That is, since the open space S is positioned on the humidity control space 114 as shown in FIG. 3, air of the open space S may continuously receive moisture from water 400 of the humidity control space 114. Therefore, the dry external air may pass through the open space S to be changed into humidified air, and provide moisture to the bio material 300 of the culture region 112.

In particular, since the cell chip 100 of the present embodiment has a structure in which the culture region 112 is surrounded by the first wall member 130 as shown in FIG. 4, the dry external air which does not sufficiently absorb moisture may not be easily introduced from the open space S to the culture region 112. More specifically, in the present cell chip 100, most of the external air may form a humidified air layer while remaining in the humidity control space 114 due to the first wall member 130. Here, a portion of the humidified air layer may be introduced to the culture region 112. However, remaining humidified air forms a predetermined air barrier, such that the dry external air may not be easily introduced to the culture region 112.

Therefore, in the cell chip 100 according to the present embodiment, the humidity of the culture region 112 may generally be constantly maintained, regardless of an external environment.

Hereinafter, a modified form of the present embodiment will be described with reference to FIGS. 5 to 8.

According to another embodiment of the first substrate 110, the substrate 110 may be provided with a groove 116 receiving the bio material or the drug 310 as shown in FIG. 5. Here, the groove 116 may be formed in the culture region 112, and a plurality of grooves may be formed, having a predetermined distance therebetween.

In addition, according to another embodiment of the first substrate 110, a position of a bottom surface (a bottom surface of the groove 116 in FIG. 5) of the culture region 112 is lower than that of a bottom surface of the humidity control space 114. That is, a height h1 from a bottom surface of the first substrate 110 to the bottom surface of the groove 116 may be lower than that a height h2 from the bottom surface of the first substrate 110 to the bottom surface of the humidity control space 114. The structure as described above may allow relatively light dry external air to pass through the culture region 112 as it is, and relatively heavy and humidified air to remain in the culture region 112.

Meanwhile, the second substrate 120 may include a plurality of protrusions 122 as shown in FIG. 6. The protrusion 122 may be inserted into the groove 116 of the first substrate 110 in a state in which the first substrate 110 and the second substrate 120 are coupled to each other. Here, the bio material 300 may be attached or fixed to an end of the protrusion 122. Therefore, when the first substrate 110 and the second substrate 120 are coupled to each other, the bio material 300 of the protrusion 122 may be immersed in the drug 310 filled in the groove 116.

In the cell chip 100 as configured above, the bio material 300 may be reacted with the drug 310 in a state in which a predetermined humidity is maintained.

According to another embodiment of the first wall member 130, the first wall member 130 may completely surround the culture region 112 as shown in FIG. 7. Meanwhile, when the first wall member 130 completely surrounds the culture region 112, since gases (for example, oxygen and carbon dioxide) required for the bio material are not supplied into the culture region 112, at least one or more first holes 134, 135, 136, and 137 may be formed in the first wall member 130. Here, the number of the first holes 134, 135, 136, and 137, and positions thereof may be changed according to a size of the substrate and a usage thereof.

According to another embodiment of the first wall member, the first wall member may be divided into a plurality of non-continuous members 130 and 131 as shown in FIG. 8. That is, the first wall members 130 and 131 may partially surround the culture region 112. Therefore, first open parts 150 and 151 allowing the culture region 112 and the humidity control space 114 to be in communication with each other may be formed between the divided first wall member 130 and the first wall member 131. Here, the sizes of the open parts 150 and 151 may be controlled according to the usage and the usage environment of the cell chip 100.

Since air circulation between the culture region 112 and the humidity control space 114 is relatively smoothly achieved in the cell chip 100 as configured above, the bio material or the drug may be stored therein for an extended period of time.

For reference, although the first wall member 130 is formed on the first substrate 110 in the above-mentioned embodiment; the first wall member 130 may be formed on the second substrate 120 as needed. In addition, a portion of the first wall member 130 may be formed on the first substrate 110 and a remaining portion thereof may be formed on the second substrate 120.

A cell chip according to another embodiment of the present invention will be described with reference to FIGS. 9 to 12. For reference, in another embodiment, the same reference numerals will be used to describe the same components as those of the embodiment, and therefore, a detailed description of these components will be omitted.

The cell chip 100 according to another embodiment of the present invention may be differentiated from the aforementioned embodiment in that the cell chip 100 of another embodiment further includes a second wall member 140.

In the present embodiment, the first substrate 110 may include the first wall member 130 and the second wall member 140. The first wall member 130 may be formed so as to surround the culture region 112, and the second wall member 140 may be formed so as to surround the humidity control space 114. Therefore, the first substrate 110 may be provided with an air passage extended along the humidity control space 114 in a lengthwise manner by two wall members 130 and 140. Here, since the first wall member 130 and the second wall member 140 are provided with the first cut part 132 and the second cut part 142, respectively, such that the air passage may be formed from the second cut part 142 to the culture region 112.

In the cell chip 100 as configured above, air introduced through the second cut part 142 moves between two wall members 130 and 140, and sufficient moisture is supplemented from the water 400 of the humidity control space 114 to thereby be effective in maintaining the humidity of the culture region 112 to a high degree (please see FIGS. 10 and 11).

Meanwhile, the first wall members 130 and 131 and the second wall members 140 and 141 may be divided into non-continuous portions as shown in FIG. 12. Here, the second wall members 140 and 141 may be disposed so as to close the first open parts 150 and 151 formed between the first wall members 130 and 131. Therefore, air introduced through the second open parts 160 and 161 between the second wall members 140 and 141 may move along the humidity control space 114 to some extent and introduced to the first open parts 150 and 151.

Since air circulation between the culture region 112 and the humidity control space 114 is relatively smoothly achieved in the cell chip 100 as configured above, the bio material or the drug may be stored for an extended period of time.

For reference, both of the first wall member 130 and the second wall member 140 are formed on the first substrate 110 in the above-mentioned embodiment; however, the first wall member 130 may be formed on the first substrate 110 and the second wall member 140 may be formed on the second substrate 120, as needed. Otherwise, the second wall member 140 may be formed on the first substrate 110 and the first wall member 130 may be formed on the second substrate 120. In other words, a portion of the first wall member 130 and a portion of the second wall member 140 may be formed on the first substrate 110, and a remaining portion of the first wall member 130 and a remaining portion of the second wall member 140 may be formed on the second substrate 120.

Hereinafter, a container for storing the cell chip according to an embodiment of the present invention will be described with reference to FIGS. 13 to 16.

The container for storing the cell chip 200 according to the embodiment of the present invention may include a bottom member 210 and a cover member 220. Here, the bottom member 210 and the cover member 220 may be coupled to each other in a vertical direction, and they may be separated from each other as needed.

The bottom member 210 may receive at least one cell chip 102. To this end, one surface of the bottom member 210 may be provided with at least one mounting space 212. Here, the mounting space 212 may have the same size as the cell chip 102 so as to receive only one cell chip 102. Otherwise, the mounting space 212 may have the same size as that of a structure in which a plurality of cell chips 102 are connected to each other in a vertical or horizontal direction so as to receive the plurality of cell chips 102 at the same time.

The bottom member 210 may further include the humidity control space 214. The humidity control space 214 may be formed at the circumference of the mounting space 212. More specifically, the humidity control space 214 may surround the plurality of respective mounting spaces 212. The humidity control space 214 as described above may be filled with water for controlling humidity. Therefore, surroundings of the mounting space 212 may be maintained at a constant humidity by water in the humidity control space 214.

The bottom member 210 may further include the first wall member 230. The first wall member 230 may surround the respective mounting spaces 212. Therefore, the bottom member 210 may be divided into the mounting space 212 and the humidity control space 214 by the first wall member 230.

The first wall member 230 may be provided with at least one or more first cut parts 232 and 233. Here, the first cut parts 232 and 233 may be used as an air passage to be in communication with the interior and the exterior environment of the mounting space 212.

The cover member 220 may be coupled to the bottom member 210. More specifically, the cover member 220 may be disposed above the bottom member 210 and cover one surface of the bottom member 210. To this end, the cover member 220 may have the generally same size as the bottom member 210.

Meanwhile, the cover member 220 may have a predetermined open space S in a state in which the cover member 220 and the bottom member 210 are coupled to each other. That is, in the state in which the bottom member 210 and the cover member 220 are coupled to each other, an edge of the bottom member 210 and an edge of the cover member 220 may have a predetermined open space S.

The open space S may be formed on the humidity control space 214. The open space S formed as described above may be used as a buffer space preventing a dry external air from being directly introduced to the mounting space 212. That is, since the open space S is positioned on the humidity control space 214 as shown in FIG. 15, air of the open space S may continuously receive moisture from water 400 of the humidity control space 214. Therefore, the dry external air may pass through the open space S to be changed into a humidified air, and thus, moisture may be provided to the cell chip 102 disposed in the mounting space 212.

In particular, since the container for storing the cell chip 200 of the present embodiment has a structure in which the mounting space 212 is surrounded by the first wall member 230 as shown in FIG. 15, the dry external air may not be easily introduced to the mounting space 212. Therefore, in the container for storing the cell chip 200 according to the present embodiment, internal humidity of the mounting space 212 may be constantly maintained regardless of an external environment.

Meanwhile, the cover member 220 may be provided with the second wall member 240. The second wall member 240 formed as described above may surround the humidity control space 214. Therefore, the container 200 for storing the cell chip may be provided with an air passage extended along the humidity control space 214 by two wall members 230 and 240.

Here, the first wall member 230 and the second wall member 240 are provided with the first cut part 232 and the second cut part 242, respectively, such that the air passage may be formed from the second cut part 242 to the mounting space 212.

Since the container for storing the cell chip 200 as configured above has a space in which predetermined moisture is provided to the external air, a phenomenon in which the dry air is introduced to the mounting space 212 may be significantly reduced.

For reference, the first wall member 230 is formed on the bottom member 210 and the second wall member 240 is formed on the cover member 220 in the above-mentioned embodiment; however, both of the first wall member 230 and the second wall member 240 may be formed on the bottom member 210 or the cover member 220 as needed. Otherwise, the second wall member 240 may be formed on the bottom member 210 and the first wall member 230 may be formed on the cover member 220. In other words, a portion of the first wall member 230 and a portion of the second wall member 240 may be formed on the bottom member 210, and the remaining portion of the first wall member 230 and the remaining portion of the second wall member 240 may be formed on the cover member 220.

As set forth above, according to the embodiments of the present invention, the phenomenon that the bio material of the cell chip is dried may be effectively suppressed.

While the present invention has been shown and described in connection with the embodiments, it will be apparent to those skilled in the art that modifications and variations can be made without departing from the spirit and scope of the invention as defined by the appended claims.

What is claimed is:

1. A cell chip comprising:
a first substrate provided with a culture region receiving a bio-material and a humidity control space storing water for maintenance of humidity;
a second substrate coupled to the first substrate; and
a first wall member surrounding the culture region.

2. The cell chip of claim 1, wherein the first wall member is formed on the first substrate.

3. The cell chip of claim 1, wherein the first wall member is formed on the second substrate.

4. The cell chip of claim 1, wherein the humidity control space is formed along an edge of the culture region in a lengthwise manner.

5. The cell chip of claim 1, wherein the humidity control space is disposed in a position higher than that of the culture region.

6. The cell chip of claim 1, wherein the first wall member is provided with at least one hole allowing the humidity control space and the culture region to be in communication with each other.

7. The cell chip of claim 1, wherein the first wall member partially surrounds the culture region.

8. The cell chip of claim 1, wherein the culture region is configured of at least one groove receiving the bio-material.

9. The cell chip of claim 1, further comprising a second wall member surrounding the humidity control space.

10. The cell chip of claim 9, wherein the second wall member is formed on the first substrate.

11. The cell chip of claim 9, wherein the second wall member is formed on the second substrate.

12. The cell chip of claim 9, wherein the second wall member is provided with at least one hole allowing the exterior environment and the humidity control space to be in communication with each other.

13. The cell chip of claim 9, wherein the second wall member partially surrounds the culture region.

14. The cell chip of claim 13, wherein the first wall member partially surrounds the culture region, and the second wall member surrounds a remaining portion of the culture region.

15. A container for storing a cell chip comprising:
a bottom member provided with a mounting space receiving at least one cell chip and a humidity control space storing water for maintenance of humidity;
a cover member coupled with the bottom member; and
a first wall member surrounding the mounting space.

16. The container for storing a cell chip of claim 15, wherein the first wall member is formed on the bottom member.

17. The container for storing a cell chip of claim 15, wherein the first wall member is formed on the cover member.

18. The container for storing a cell chip of claim 15, wherein the humidity control space is formed along an edge of the mounting space in a lengthwise manner.

19. The container for storing a cell chip of claim 15, the humidity control space is disposed in a position higher than that of the mounting space.

20. The container for storing a cell chip of claim 15, wherein the first wall member is provided with at least one hole allowing the humidity control space and the mounting space to be in communication with each other.

21. The container for storing a cell chip of claim 15, wherein the first wall member partially surrounds the mounting space.

22. The container for storing a cell chip of claim 15, further comprising a second wall member surrounding the humidity control space.

23. The container for storing a cell chip of claim 22, wherein the second wall member is formed on the bottom member.

24. The container for storing a cell chip of claim 22, wherein the second wall member is formed on the cover member.

25. The container for storing a cell chip of claim 22, wherein the second wall member is provided with at least one hole allowing the exterior environment and the humidity control space to be in communication with each other.

26. The container for storing a cell chip of claim 22, wherein the second wall member partially surrounds the culture region.

27. The container for storing a cell chip of claim 26, wherein the first wall member partially surrounds the culture region, and the second wall member surrounds a remaining portion of the culture region.
